(19) United States
(12) Patent Application Publication
(10) Pub. No.: US 2007/0116687 A1
(43) Pub. Date: May 24, 2007

(54) Method of modulating cellular transmigration and agents for use therein

(75) Inventors: Yeesim Khew-Goodall, Netherby (AU); Mathew Alexander Vadas, Stirling (AU); Jennifer Ruth Gamble, Stirling (AU); Brian Stein, Rose Park (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: Medvet Science PTY, Ltd.

(21) Appl. No.: 10/556,525

(22) PCT Filed: May 13, 2004
(86) PCT No.: PCT/AU04/00627

§ 371 Date: Nov. 27, 2006

(30) Foreign Application Priority Data
May 13, 2003 (AU) 2003902295
Aug. 12, 2003 (AU) 2003904284

(51) Int. Cl. A61K 48/00; A61K 39/395

(52) U.S. Cl. 424/93.21; 424/146.1; 514/44

(57) ABSTRACT

The present invention relates generally to a method of modulating cellular transendothelial migration and to agents useful for same. More particularly, the present invention relates to a method of modulating leukocyte extravasation by modulating an endothelial cell intracellular ERK (extracellular regulated kinase)-dependent signalling mechanism. The method of the present invention is useful, inter alia, in the treatment and/or prophylaxis of conditions characterised by aberrant, unwanted or otherwise inappropriate transendothelial cells migration, in particular, inflammatory conditions which are characterised by inappropriate leukocyte and, in particular, neutrophil transendothelial migration.

### FIELD OF THE INVENTION

The present invention relates generally to a method of modulating cellular transendothelial migration and to agents useful for same. More particularly, the present invention relates to a method of modulating leukocyte extravasation by modulating an endothelial cell intracellular ERK (extracellular regulated kinase)-dependent signalling mechanism. The method of the present invention is useful, inter alia, in the treatment and/or prophylaxis of conditions characterised by aberrant, unwanted or otherwise inappropriate transendothelial cells migration, in particular, inflammatory conditions which are characterised by inappropriate leukocyte and, in particular, neutrophil transendothelial migration.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

One of the essential functions of the endothelial cell lining is to maintain the essentially impermeable nature of the blood vessel controlling the passage of solutes and inflammatory cells from the circulation to the tissues. Endothelial cell hyper-permeability is a characteristic of blood vessels in many pathologies. For example, newly formed micro-vessels in tumours are highly permeable. Indeed, such hyper-permeability allows the deposition of fibrin in tumours that supports and promotes cell adhesion and migration, essential steps in the angiogenic response (Dvorak, H. F., Harvey, V. S., Estrella, P., Brown, L. F., McDonagh, J., Dvorak, A. M. (1987) Lab Invest. 57:673-86; Dvorak, H. F., Brown, L. F., Detmar, M., Dvorak, A. M. (1995) Am J Pathol. 146:1029-39). In chronic inflammatory states such as in rheumatoid arthritis and atherosclerosis, vessel hyper-permeability allows increased transmigration of inflammatory cells across the activated endothelium. A number of factors have previously been described which promote endothelial cell leakiness, for example, thrombin, tumour necrosis factor and vascular endothelial cell growth factor (VEGF). These appear to act by inducing changes in junctional molecules such as PECAM-1 and VE-cadherin or their associated signalling molecules, such as the catenins.

Leukocyte extravasation is a multistep process involving tethering, rolling, firm adhesion and finally transendothelial migration into the sub-endothelial space (Butcher, E. C., Cell 67:1033-1036, 1991; Springer, T. A., 1994, Cell 76:301-314). The mechanisms by which tethering, rolling and firm adhesion occur are relatively well-characterised, particularly in comparison to the current understanding of the later stages of this process, being the mechanisms by which leukocytes traverse the endothelium. Under non-inflammatory conditions, the endothelium has low permeability to leukocytes but when an inflammatory response is initiated, the paracellular permeability of the endothelium is increased to enable leukocytes to pass in between endothelial cells.

It is now widely accepted that most leukocyte extravasation occurs at interendothelial junctions and that cell-cell adhesion receptors not only maintain the architecture of the endothelium but also play a role in regulating vascular permeability (Dejana, E. et al., 1995, FASEB J 9:910-918; Dejana, E. et al., 2000, Int. J. Dev. Biol. 44:743-748). Of particular relevance to regulating leukocyte extravasation are the homophilic adhesion receptors, vascular endothelial (VE)-cadherin (Del Maschio, A. et al., 1996, J Cell Biol 135:497-510; Allport, J. R. et al., 1997, J Exp. Med. 186:517-527; Allport, J. R. et al. 2000, J Cell Biol 148:203-216), an adherence junction protein, and platelet-endothelial cell adhesion molecule-1 (PECAM-1) (Muller, W. A. et al., 1993, J Exp. Med. 178:449-460; Newman, P. J. 1997, J. Clin. Invest 100:S25-S29; Muller, W. A. et al., 1999, J Leukoc. Biol 66:698-704; Nakada M. T. et al., 2000, J Immunol. 164:452-462)

In order for leukocytes to transmigrate across a fully sealed endothelium, adhesion between endothelial cells has to be transiently released to create a gap for the leukocytes to pass through. In addition, it is also possible that some form of transient adhesion between the endothelial cells and leukocytes is established as the leukocyte migrates through. One would therefore predict that mechanisms which disrupt interendothelial adhesion are set into action either when endothelial cells become activated by inflammatory cytokines or when activated leukocytes marginate and interact with the endothelium. Some of the mechanisms elucidated to date include the cleavage of adhesion receptors by elastase bound to the surface of leukocytes (Cepinskas, G. et al., 1997, Circ. Res. 81:618-626; Cepinskas, G. et al., 1999, J Cell Sci 112 (Pt 12):1937-1945) and activation of endothelial intracellular signalling pathways by adherent leukocytes (Bianchi, E. et al., 1997, Immunol. Today 18:586-591). However, surface-bound elastase is unlikely to be a universal or major mechanism because monocytes and some monocytic cell lines that do not have surface-bound elastase can aptly transmigrate (Allport et al., 1997, supra). Furthermore, there is now evidence that adhesion molecules such as VE-cadherin move away from the site of leukocyte passage, rather than being disrupted, whereas endothelial-endothelial PECAM-1 adhesion is released to enable the leukocyte to pass through (Shaw, S. K. et al. 2001, J Immunol. 167:2323-2330; Su, W. H. et al., 2002, Blood 100:3597-3603). Both these adhesion molecules are found to be displaced very transiently and returned to their earlier positions within a short time after the passage of the leukocyte; the period is too short for de novo synthesis of intact receptors to replace the cleaved ones (Su et al., 2000, supra).

Activation of endothelial intracellular signalling pathways therefore is likely to be essential for releasing PECAM-PECAM interaction or moving VE-cadherin away to enable the paracellular passage of leukocytes. It has been reported that leukocyte adherence leads to increases of endothelial intracellular Ca⁺⁺ that is essential for leukocyte transmigration to proceed (Huang, A. J. et al., 1993, J Cell Biol 120:1371-1380; Su, W. H. et al., 2000, Blood 96:3816-3822). Activation of myosin light chain kinase (MLCK) has also been observed to be essential for leukocyte transmigration (Hixenbaugh, E. A., et al., 1997, Am. J Physiol 273:H981-H988; Saito, H. et al., 1998, J Immunol. 161:1533-1540). However, the signals which regulate endothelial cell permeability are far from having been fully defined.

Since the traversal of leukocytes across the endothelial barrier and into the tissue space is an integral component of an inflammatory response induced by infection or injury, there is an urgent need to elucidate these signalling mechanisms in order to facilitate the development of therapeutic and/or prophylactic strategies directed to treating conditions characterised by aberrant or otherwise unwanted endothelial transmigration.

In work leading up to the present invention, it has been surprisingly determined that activation of the migration activated protein (MAP) kinases ERK 1 and/or ERK 2, in the endothelium, is essential for neutrophils to traverse the endothelial barrier. These findings support the notion that endothelial transmigration is a complex process involving the functioning of multiple parallel signalling pathways and now facilitate the rational design of methodology directed to modulating cellular transendothelial migration, in particular neutrophil extravasation, by regulating the functioning of ERK 1 and/or ERK 2.

### SUMMARY OF THE INVENTION

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The present invention is not to be limited in scope by the specific embodiments described herein, which are intended for the purposes of exemplification only. Functionally-equivalent products, compositions and methods are clearly within the scope of the invention, as described herein.

As used herein, the term “derived from” shall be taken to indicate that a particular integer or group of integers has originated from the species specified, but has not necessarily been obtained directly from the specified source.

One aspect of the present invention is directed to a method of modulating cellular transendothelial cell migration, said method comprising modulating endothelial cell ERK functional activity wherein upregulating ERK activity to a functionally effective level upregulates said migration and downregulating said activity to a functionally ineffective level downregulates said migration.

Another aspect of the present invention provides a method of modulating cellular transendothelial cell migration, which endothelial cells are vascular endothelial cells, said method comprising modulating said endothelial cell ERK functional activity wherein upregulating ERK activity to a functionally effective level upregulates said migration and downregulating said activity to a functionally ineffective level downregulates said migration.

Yet another aspect of the present invention provides a method of modulating leukocyte extravasation, said method comprising modulating vascular endothelial cell ERK functional activity wherein upregulating ERK activity to a functionally effective level upregulates said extravasation and downregulating said activity to a functionally ineffective level downregulates said extravasation.

Still another aspect of the present invention provides a method of modulating neutrophil extravasation, said method comprising modulating vascular endothelial cell ERK functional activity wherein upregulating ERK activity to a functionally effective level upregulates said extravasation and downregulating said activity to a functionally ineffective level downregulates said extravasation.

Yet still another aspect of the present invention is directed to a method of modulating cellular transendothelial cell migration in a mammal, said method comprising modulating endothelial cell ERK functional activity in said mammal wherein upregulating ERK activity to a functionally effective level upregulates said migration and down-regulating ERK activity to a functionally ineffective level downregulates said migration.

In still yet another aspect there is provided the method of modulating cellular transendothelial cell migration in a mammal, which endothelial cells are vascular endothelial cells, said method comprising modulating endothelial cell ERK functional activity wherein upregulating ERK activity to a functionally effective level upregulates said migration and downregulating ERK activity to a functionally ineffective level downregulates said migration.

A further aspect of the present invention provides a method of upregulating cellular transendothelial cell migration in a mammal, said method comprising administering to said mammal an effective amount of an agent for a time and under conditions sufficient to induce a functionally effective level of ERK.

In another further aspect there is provided a method of upregulating cellular transendothelial cell migration in a mammal, said method comprising administering to said mammal an effective amount of ERK for a time and under conditions sufficient to induce a functionally effective level of ERK.

In still another further aspect there is provided a method of upregulating cellular transendothelial cell migration in a mammal, said method comprising administering to said mammal an effective amount of a nucleotide sequence encoding ERK for a time and under conditions sufficient to induce a functionally effective level of ERK.

In yet another further aspect there is provided a method of downregulating cellular transendothelial cell migration in a mammal, said method comprising administering to said mammal an effective amount of an agent for a time and under conditions sufficient to induce a functionally ineffective level of ERK.

Still yet another aspect of the present invention provides a method for the treatment and/or prophylaxis of a condition characterised by aberrant, unwanted or otherwise inappropriate cellular transendothelial cell migration in a mammal, said method comprising modulating the functional activity of ERK wherein upregulating ERK activity to a functionally effective level upregulates said cellular transendothelial cell migration and down-regulating ERK activity to a functionally ineffective level downregulates said cellular transendothelial cell migration.

In yet still another further aspect there is provided a method for the treatment and/or prophylaxis of a condition characterised by unwanted cellular transendothelial cell migration in a mammal, said method comprising administering to said mammal an effective amount of an agent for a time and under conditions sufficient to induce a functionally ineffective level of ERK.

Another aspect of the present invention relates to the use of an agent capable of modulating the functionally effective level of ERK in the manufacture of a medicament for the regulation of cellular transendothelial cell migration in a mammal wherein upregulating ERK activity to a functionally effective level upregulates said cellular transendothelial cell migration and downregulating ERK activity to a functionally ineffective level downregulates said cellular transendothelial cell migration.

In still another aspect the present invention relates to the use of ERK or a nucleic acid encoding ERK in the manufacture of a medicament for the regulation of cellular transendothelial cell migration wherein upregulating ERK to a functionally level upregulates said cellular transendothelial cell migration.

In yet another further aspect, the present invention contemplates a pharmaceutical composition comprising the modulatory agent as hereinbefore defined and one or more pharmaceutically acceptable carriers and/or diluents.

### BRIEF DESCRIPTION OF THE DRAWINGS

FIG. 1 is a graphical representation of inhibitors that act on MEK inhibit neutrophil transmigration across endothelium. A, Confluent HUVEC monolayers plated (5×10⁴ cells/well) on Transwells were either unstimulated or activated with TNFα (4 ng/ml) for 4 hours. Thirty min prior to the assay some monolayers were treated with PD98059 (45 μM) or DMSO control. Neutrophils (5×10⁵ cells/well) were added with either PD98059 or DMSO. Transmigration was assayed across TNFα-activated endothelium (TNF) and unstimulated endothelium with (fMLP) or without (Nil) a gradient of 10 nM fMLP. The assays were carried out in triplicate and 1 representative of 5 experiments shown. An ANOVA was performed looking for an effect of PD98059 compared to DMSO on transmigration: in this experiment p<0.0001 and in the overall series p<0.0001. B, Endothelial monolayers were treated as in A, but the concentration of PD98059 was varied as indicated. The data are presented as percentage of transmigration relative to that across unstimulated endothelium. One representative experiment of 7, each performed in triplicate, is shown. ANOVA performed looking for an effect of PD98059 compared to DMSO on transmigration showed p<0.001 for TNFα-activated endothelium and p<0.001 for an fMLP gradient). C, Endothelial cells on Transwells were left unstimulated or activated with TNFα as in A. Using the standard protocol, U0126 or its solvent, DMSO, were added to the monolayers 30 min prior to the experiment, and then added with the neutrophils. The results from one of two experiments, each performed in triplicate, is shown. ANOVA performed showed p<0.001 when looking for an effect of U0126 compared to DMSO on transmigration.

FIG. 2 is a graphical representation demonstrating that PD98059 did not inhibit neutrophil chemotaxis or adhesion to endothelium. A, Chemotaxis assay using neutrophils pretreated for 20 min at room temperature with 30 μM PD98059 or DMSO. Chemotaxis was stimulated by the presence of 10 nM fMLP, or 1 nM IL-8 in the lower chamber; medium alone served as a control (nil). The data represent one of three experiments, each performed in triplicate. ANOVA looking for an effect of PD98059 compared to DMSO on chemotaxis gave p=0.359. B, Confluent HUVEC monolayers were stimulated with TNFα for 4 h, with PD98059 or DMSO added in the final 30 min. The extent of neutrophil adhesion to the endothelium was determined using a standard adhesion assay. The data is presented as the percentage of neutrophil remaining adherent relative to that added (% adhesion). The results represent one of three experiments, each performed in triplicate. (p=0.238, t-test comparing DMSO+TNF with PD+TNF).

FIG. 3 is an image indicating that the presence of neutrophils is essential for activation of endothelial Erk. A, Endothelial cells treated with 0.4 ng/ml TNFα (TNF), 30 ng/ml interleukin-4 (IL-4), 20 ng/ml oncostatin-M (OsM), 100 ng/ml PMA, or medium alone (Nil) for 15 minutes were assayed for Erk activation. Western blots shown were carried out using an anti-phospho-Erk Ab to detect activated Erk. Equal amounts of protein were loaded onto each lane. B, HUVEC were activated with TNFα for 4 hours or left unstimulated. After washing, either medium containing IL-8 (−) or neutrophils pre-treated with 1 nM IL-8 (+) were added at a 10:1 ratio of neutrophils to HUVEC and Erk activation assayed after 15 min at 37° C. Western blots shown were carried out using antibodies to phospho-Erk (upper panel) or reprobed with an anti-Erk polyclonal antibody after membranes were stripped (lower panel).

FIG. 4 is an image indicating that neutrophil adhesion is not a requirement for endothelial Erk activation. A, Neutrophils, untreated or incubated with a β₂ integrin functional blocking antibody, TS1/18 (50 μg/ml), for 20 min at room temperature were added to HUVEC in the presence of 1 nM fMLP for 15 min. HUVEC monolayers were then analysed for Erk activation by Western blotting with an anti-phospho-Erk Ab. B, Neutrophils, untreated or treated with the TS1/18 Ab as in A, were added to 96-well tissue culture dishes in the presence or absence of 1 nM fMLP and neutrophil adhesion was determined. The data is presented as the percentage of neutrophil remaining adherent relative to that added (% adhesion). By ANOVA comparing +/−TS1/18, p=0.0002, n=3.

FIG. 5 is an image indicating that conditioned media from chemoattractant-stimulated neutrophils activate Erk in endothelial cells. Resting HUVEC monolayers incubated with medium (nil), neutrophils (N) or neutrophil conditioned medium (CM), were analysed for Erk activation by Western blotting with an anti-phospho-Erk Ab. A, Neutrophils were stimulated for 15 minutes with 1 nM fMLP and then divided into two aliquots. One aliquot was centrifuged and the top 2/3 taken, carefully avoiding the cellular pellet, and added as conditioned medium (CM). The other aliquot was resuspended and 2/3 taken and added as the equivalent amount of neutrophil preparation (N). B. Conditioned media from neutrophils treated with 1 or 100 nM fMLP (prepared as in A) were added to HUVEC either undiluted (neat) or after diluting 1 in 3 (1/3) in medium. C, Conditioned media from unstimulated neutrophils or neutrophils stimulated with 10 nM IL-8 for either 15 or 45 minutes were added to HUVEC monolayers. Lower panel shows membrane re-blotted with an anti-Erk Ab after stripping.

### DETAILED DESCRIPTION OF THE INVENTION

The present invention is predicated, in part, on the determination that cellular, in particular neutrophil, transendothelial migration is critically dependent on the activation of the MAP kinases ERK 1 and/or ERK 2. This is a surprising finding when considered in light of the facts that it was not known that endothelial cell ERK activation was associated with neutrophil transmigration nor have many of the signalling pathways that mediate the late steps in transmigration been elucidated. This development now permits the rational design of therapeutic and/or prophylactic methods for treating conditions characterised by aberrant or unwanted cellular transendothelial migration.

Accordingly, one aspect of the present invention is directed to a method of modulating cellular transendothelial cell migration, said method comprising modulating endothelial cell ERK functional activity wherein upregulating ERK activity to a functionally effective level upregulates said migration and downregulating said activity to a functionally ineffective level downregulates said migration.

Reference to “endothelial cell” should be understood as a reference to the endothelial cells which line the blood vessels, lymphatics or other serous cavities such as fluid filled cavities. The phrase “endothelial cells” should also be understood as a reference to cells which exhibit one or more of the morphology, phenotype and/or functional activity of endothelial cells and is also a reference to mutants or variants thereof. “Variants” include, but are not limited to, cells exhibiting some but not all of the morphological or phenotypic features or functional activities of endothelial cells at any differentiative stage of development. “Mutants” include, but are not limited to, endothelial cells which have been naturally or non-naturally modified such as cells which are genetically modified.

It should also be understood that the endothelial cells of the present invention may be at any differentiative stage of development. Accordingly, the cells may be immature and therefore functionally incompetent in the absence of further differentiation. In this regard, highly immature cells such as stem cells, which retain the capacity to differentiate into endothelial cells, should nevertheless be understood to satisfy the definition of “endothelial cell” as utilised herein due to their capacity to differentiate into endothelial cells under appropriate conditions. Preferably, the subject endothelial cell is a vascular endothelial cell.

Accordingly, there is more particularly provided a method of modulating cellular transendothelial cell migration, which endothelial cells are vascular endothelial cells, said method comprising modulating said endothelial cell ERK functional activity wherein upregulating ERK activity to a functionally effective level upregulates said migration and downregulating said activity to a functionally ineffective level downregulates said migration.

Reference to “transendothelial cell migration” should be understood as a reference to the migration of a cell from one side of a tissue comprising endothelial cells (“endothelium”) through to the other side of this tissue. Without limiting the present invention to any one theory or mode of action, such migration, for example leukocyte extravasation, is a multistep process involving rolling, tethering, adhesion to the endothelium and then migration across the endothelium. More specifically, and in the context of leukocyte extravasation, under normal conditions, leukocytes are generally restricted to the center of blood vessels, where the flow is fastest. At sites of inflammation, where the vessels are dilated, the slower blood flow allows the leukocytes to move out of the center of the blood vessel and to interact with the vascular endothelium. In addition to these changes, there is an increase in vascular permeability, leading to the local accumulation of fluid—hence the swelling and pain—as well as the accumulation of immunoglobulins, complement, and other blood proteins in the tissue. A further effect of these mediators on endothelium is to induce the expression of adhesion molecules that bind to the surface of circulating monocytes and polymorphonuclear leukocytes and greatly enhance the rate at which these phagocytic cells migrate across local small blood vessel walls into the tissues. During an inflammatory response, the induction of adhesion molecules on the local blood vessels, as well as inducing changes in the adhesion molecules expressed on the leukocytes, recruit large numbers of circulating phagocytic cells, mainly neutrophils and monocytes, into the site of an infection.

The first step in leukocyte extravasation involves the reversible binding of leukocytes to vascular endothelium through interactions between adhesion receptors induced on the endothelium and their carbohydrate ligands on the leukocyte. This binding cannot anchor the cells against the shearing force of the flow of blood and instead they roll along the endothelium, continually making and breaking contact. The binding does, however, allow stronger interactions, which occur as a result of the induction of further adhesion molecules on the endothelium and the activation of counter receptors on the leukocyte. Tight binding between these molecules arrests the rolling and allows the leukocyte to squeeze between the endothelial cells forming the wall of the blood vessel (extravasate). Adhesion between molecules expressed on both leukocyte and at the junction of the endothelial cells, is also thought to contribute to diapedesis. In the context of the present invention, said cellular transendothelial cell migration is preferably leukocyte extravasation.

According to this preferred embodiment, there is provided a method of modulating leukocyte extravasation, said method comprising modulating vascular endothelial cell ERK functional activity wherein upregulating ERK activity to a functionally effective level upregulates said extravasation and downregulating said activity to a functionally ineffective level downregulates said extravasation.

Reference to a “leukocyte” should be understood as a reference to any white blood cell including lymphocytes, monocytes, polymorphonuclear leukocytes and mutants and variants thereof. Analogous to the definition provided earlier in the context of endothelial cells, reference to “leukocyte” should be understood as a reference to a leukocyte at any differentiative stage of development. Preferably, the subject leukocyte is a neutrophil.

The present invention therefore still more preferably provides a method of modulating neutrophil extravasation, said method comprising modulating vascular endothelial cell ERK functional activity wherein upregulating ERK activity to a functionally effective level upregulates said extravasation and downregulating said activity to a functionally ineffective level downregulates said extravasation.

Reference to “ERK” should be understood as a reference to all forms of these proteins (e.g. ERK 1 and ERK 2) and to functional derivatives, homologues, analogues, chemical equivalents or mimetics thereof. This includes, for example, any isoforms which arise from alternative splicing of the subject ERK or mutants or polymorphic variants of these proteins.

Without limiting the present invention to any one theory or mode of action, it is thought that multiple parallel signalling pathways are activated within the endothelium during transmigration. The pathway of the present invention is thought to involve the phosphorylation of the MAP kinases ERK 1 and/or ERK 2 by MEK (Mitogen activated protein kinase/extracellular regulated kinase kinase). It has been still further determined that the subject ERK activation does not occur downstream of Ca⁺⁺-dependent phosphorylation of myosin light chain kinase (MLCK). This latter event is thought to be one of the above-referenced “parallel pathways” essential to the cytoskeletal remodelling events which correlate with increased endothelial cell permeability. In this regard, many of the interendothelial adhesion receptors are limited to the actin cytoskeleton either directly or through their interactions with a number of cytosolic proteins (Lampugnani, M. G. et al., 1997, Curr. Opin. Cell Biol 9:674-682). It is therefore thought that leukocyte adhesion-dependent intracellular Ca⁺⁺ fluxes activate MLCK to reorganise the cytoskeleton, leading to alterations in interendothelial adhesion receptor function that facilitate leukocyte transmigration.

With respect to the ERK-based signalling mechanism herein disclosed, although ERK activation is commonly associated with mitogenic signalling, it has a large array of substrates including a number of microtubule associated proteins (Schlesinger, T. K. et al., 1998, Front Biosci 3:D1181-D1186) which when phosphorylated by ERK result in destabilisation of the microtubules (Hoshi, M. et al., 1992, Eur. J. Biochem 203:43-52). In light of the current findings, the interaction between the microtubular and actin cytoskeletons suggests that ERK activation could also be involved in alterations to cell-cell adhesion during transmigration, although other mechanisms of action cannot be ruled out. Recent findings show that multiple cell adhesion receptors are modulated during transmigration and moreover that different mechanisms are used to regulate different receptors to increase paracellular permeability (Shaw et al., 2001, supra; Su et al., 2000, supra). It would therefore not be unexpected to find multiple signalling pathways activated to regulate different aspects of cytoskeletal function associated with cell-cell adhesion.

Still without limiting the present invention to any one theory or mode of action, it has been determined (as hereinafter discussed in more detail) that one of the triggers for activating ERK is a soluble protein factor produced by activated neutrophils. However, it should be understood that the present invention is directed to the modulation of cellular transmigration across endothelial cells, per se, irrespective of the nature of a specific stimulatory, or inhibitory, signal.

Reference to “modulating” should be understood as a reference to upregulating or downregulating the subject transendothelial cell migration. Reference to “downregulating” transendothelial cell migration should therefore be understood as a reference to preventing, reducing (e.g. slowing) or otherwise inhibiting one or more aspects of this event (for example retarding or preventing rolling, tight binding or diapedesis) while reference to “upregulating” should be understood to have the converse meaning.

Reference to ERK “functional activity” should be understood as a reference to any one or more of the activities which ERK can perform. Accordingly, reference to “modulating” ERK functional activity is a reference to either upregulating or downregulating ERK functional activity. Such modulation may be achieved by any suitable means and includes:
(i) Modulating absolute levels of the active or inactive forms of ERK (for example increasing or decreasing intracellular ERK concentrations) such that either more or less ERK is available for activation and/or to interact with its downstream targets.
(ii) Agonising or antagonising ERK such that the functional effectiveness of any given ERK molecule is either increased or decreased. For example, increasing the half life of ERK may achieve an increase in the overall level of ERK activity without actually necessitating an increase in the absolute intracellular concentration of ERK. Similarly, the partial antagonism of ERK, for example by coupling ERK to a molecule that introduces some steric hindrance in relation to the binding of ERK to its downstream targets, may act to reduce, although not necessarily eliminate, the effectiveness of ERK signalling. Accordingly, this may provide a means of downregulating ERK functioning without necessarily downregulating absolute concentrations of ERK.

In terms of achieving the up or downregulation of ERK functioning, means for achieving this objective would be well known to the person of skill in the art and include, but are not limited to:
(i) Introducing into a cell a nucleic acid molecule encoding ERK or functional equivalent, derivative or analogue thereof in order to upregulate the capacity of said cell to express ERK.
(ii) Introducing into a cell a proteinaceous or non-proteinaceous molecule which modulates transcriptional and/or translational regulation of a gene, wherein this gene may be a ERK gene or functional portion thereof or some other gene which directly or indirectly modulates the expression of the ERK gene.
(iii) Introducing into a cell the ERK expression product (in either active or inactive form) or a functional derivative, homologue, analogue, equivalent or mimetic thereof.
(iv) Introducing a proteinaceous or non-proteinaceous molecule which functions as an antagonist to the ERK expression product.
(v) Introducing a proteinaceous or non-proteinaceous molecule which functions as an agonist of the ERK expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the ERK expression product or small molecules capable of acting as agonists or antagonists. Chemical agonists may not necessarily be derived from the ERK expression product but may share certain conformational similarities. Alternatively, chemical agonists may be specifically designed to meet certain physiochemical properties. Antagonists may be any compound capable of blocking, inhibiting or otherwise preventing ERK from carrying out its normal biological function, such as molecules which prevent its activation or else prevent the downstream functioning of activated ERK (for example PD98059, U0126 or PD184352). Antagonists include monoclonal antibodies and antisense nucleic acids which prevent transcription or translation of ERK genes or mRNA in mammalian cells. Modulation of expression may also be achieved utilising antigens, RNA, ribozymes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the ERK gene or functional equivalent or derivative thereof with an agent and screening for the modulation of ERK protein production or functional activity, modulation of the expression of a nucleic acid molecule encoding ERK or modulation of the activity or expression of a downstream ERK cellular target. Detecting such modulation can be achieved utilising techniques such as Western blotting, electrophoretic mobility shift assays and/or the readout of reporters of ERK activity such as luciferases, CAT and the like.

It should be understood that the ERK gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate ERK activity, at either the nucleic acid or expression product levels, or the gene may require activation—thereby providing a model useful for, inter alia, screening for agents which up regulate ERK expression. Further, to the extent that an ERK nucleic acid molecule is transfected into a cell, that molecule may comprise the entire ERK gene or it may merely comprise a portion of the gene such as the portion which regulates expression of the ERK product. For example, the ERK promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. One might also measure ERK activation directly. Without limiting the present invention to any one theory or mode of action ERK is generally activated by the thr/tyr phosphorylation through the upstream kinase MEK. It can be downregulated by dephosphorylation with MEP-1 (MAPK phosphatase-1).

In another example, the subject of detection could be a downstream ERK regulatory target, rather than ERK itself. For example, modulation of ERK activity can be detected by screening for the modulation of the functional activity in an endothelial cell. This is an example of an indirect system where modulation of ERK expression, per se, is not the subject of detection. Rather, modulation of the molecules and mechanisms which ERK regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the ERK nucleic acid molecule or expression product itself or which modulate the expression of an upstream molecule, which upstream molecule subsequently modulates ERK expression or expression product activity. Accordingly, these methods provide a mechanism of detecting agents which either directly or indirectly modulate ERK expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region and/or its entry to a cell.

The subject proteinaceous or non-proteinaceous molecule may act either directly or indirectly to modulate the expression of ERK or the activity of the ERK expression product. Said molecule acts directly if it associates with the ERK nucleic acid molecule or expression product to modulate expression or activity, respectively. Said molecule acts indirectly if it associates with a molecule other than the ERK nucleic acid molecule or expression product which other molecule either directly or indirectly modulates the expression or activity of the ERK nucleic acid molecule or expression product, respectively, for example, modulating the functioning of MEK. Examples of agents which function indirectly by acting on upstream kinases include PD98059, U0126 and PD184352. Accordingly, the method of the present invention encompasses the regulation of ERK nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to upregulation or downregulation.

“Derivatives” of the molecules herein described (for example ERK or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, ERK or derivative thereof may be fused to a molecule to facilitate its entry into a cell. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, Si RNAs, PCR primers, antisense molecules, molecules suitable for use in cosuppression and fusion of nucleic acid molecules. Derivatives of nucleic acid sequences also include degenerate variants.

A “variant” or “mutant” of ERK should be understood to mean molecules which exhibit at least some of the functional activity of the form of ERK of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of ERK which exhibits similar and suitable functional characteristics to that of the ERK which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (e.g., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing ERK analogues which exhibit properties such as more potent pharmacological effects. ERK or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of ERK, it may also be desirable to identify and utilise molecules which function agonistically or antagonistically to ERK in order to up or downregulate the functional activity of ERK in relation to modulating endothelial cell function. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of ERK capable of acting as agonists or antagonists. Chemical agonists may not necessarily be derived from ERK but may share certain conformational similarities. Alternatively, chemical agonists may be specifically designed to mimic certain physiochemical properties of ERK. Antagonists may be any compound capable of blocking, inhibiting or otherwise preventing ERK from carrying out its normal biological functions. Antagonists include monoclonal antibodies specific for ERK or parts of ERK.

Analogues of ERK or of ERK agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Reference herein to attaining either a “functionally effective level” or “functionally ineffective level” of ERK should be understood as a reference to attaining that level of functionally active ERK at which modulation of transendothelial cell migration can be achieved, whether that be upregulation or downregulation. In this regard, it is within the skill of the person of skill in the art to determine, utilising routine procedures, the threshold level of functionally active ERK expression above which transendothelial cell migration can be upregulated and below which this activity is downregulated. For example, suitable for use in this regard is any method which regulates the phosphorylation status or the cellular localisation of ERK, as would any method which is based on the alteration of RNA synthesis of ERK (for example, antisense constructs, DNAzymes or RNAi could change the levels of proteins). It should be understood that reference to an “effective level” means the level necessary to at least partly attain the desired response. The amount will vary depending on the health and physical condition of the cellular population and/or individual being treated, the taxonomic group of the cellular population and/or individual being treated, the degree of up or downregulation which is desired, the formulation of the composition which is utilised, the assessment of the medical situation and other relevant factors. Accordingly, it is expected that this level may vary between individual situations, thereby falling in a broad range, which can be determined through routine trials.

The method of the present invention contemplates the modulation of transendothelial cell migration in both in vitro and in vivo. Although the preferred method is to treat an individual in vivo it should nevertheless be understood that it may be desirable that the method of the invention may be applied in an in vitro environment, for example to provide an in vitro model of leukocyte extravasation. In another example the application of the method of the present invention in an in vitro environment may extend to providing a readout mechanism for screening technologies such as those hereinbefore described. That is, molecules identified utilising these screening techniques can be assayed to observe the extent and/or nature of their functional effect on endothelial cells which have been functionally modulated according to the method of the present invention.

Although the preferred method is to downregulate, extravasation (for example in order to downregulate the progression of an inflammatory response), it should be understood that there may also be circumstances in which it is desirable to upregulate transendothelial cell migration, such as vascular extravasation. For example, in some circumstances it can be desirable to upregulate an inflammatory response, for example, where infection by a pathogen or microbe has occurred.

Accordingly, another aspect of the present invention is directed to a method of modulating cellular transendothelial cell migration in a mammal, said method comprising modulating endothelial cell ERK functional activity in said mammal wherein upregulating ERK activity to a functionally effective level upregulates said migration and down-regulating ERK activity to a functionally ineffective level downregulates said migration.

More preferably, there is provided the method of modulating cellular transendothelial cell migration in a mammal, which endothelial cells are vascular endothelial cells, said method comprising modulating endothelial cell ERK functional activity wherein upregulating ERK activity to a functionally effective level upregulates said migration and down-regulating ERK activity to a functionally ineffective level downregulates said migration.

Preferably, said cellular transendothelial cell migration is vascular extravasation and even more preferably leukocyte extravasation. Most preferably, said leukocyte is a neutrophil.

Modulation of said ERK functional activity is achieved via the administration of ERK, a nucleic acid molecule encoding ERK or an agent which effects modulation of ERK activity or ERK gene expression (herein collectively referred to as “modulatory agents”). In particular, and as detailed hereinbefore, the determination of the intracellular signalling mechanism which is utilised in order to upregulate transendothelial cell migration now provides a means of modulating said activity either as a consequence of endogenous stimulation or as a means of circumventing the requirement for endogenous stimulation (this latter outcome is particularly useful in terms of the upregulation of neutrophil extravasation in the absence of neutrophil activation).

Accordingly, in one preferred embodiment there is provided the method of upregulating cellular transendothelial cell migration in a mammal, said method comprising administering to said mammal an effective amount of an agent for a time and under conditions sufficient to induce a functionally effective level of ERK.

In another preferred embodiment there is provided a method of upregulating cellular transendothelial cell migration in a mammal, said method comprising administering to said mammal an effective amount of ERK for a time and under conditions sufficient to induce a functionally effective level of ERK.

In still another preferred embodiment there is provided a method of upregulating cellular transendothelial cell migration in a mammal, said method comprising administering to said mammal an effective amount of a nucleotide sequence encoding ERK for a time and under conditions sufficient to induce a functionally effective level of ERK.

In yet another preferred embodiment there is provided a method of downregulating cellular transendothelial cell migration in a mammal, said method comprising administering to said mammal an effective amount of an agent for a time and under conditions sufficient to induce a functionally ineffective level of ERK.

In accordance with these preferred embodiments of the present invention, said endothelial cells are preferably vascular endothelial cells and said cellular transendothelial cell migration is preferably leukocyte extravasation. Most preferably, said leukocyte extravasation is neutrophil extravasation.

Reference to “induce” should be understood as a reference to achieving the desired ERK level, whether that be a functionally effective level or a functionally ineffective level. Said induction is most likely to be achieved via the upregulation or downregulation of ERK functional activity, as hereinbefore described, although any other suitable means of achieving induction are nevertheless herewith encompassed by the method of the present invention. As detailed hereinbefore, this may include, for example, the activation/overexpression of upstream regulators or switching of MKP.

A further aspect of the present invention relates to the use of the invention in relation to the treatment and/or prophylaxis of disease conditions, other unwanted conditions or normal physiology. Without limiting the present invention to any one theory or mode of action, the regulation of cellular transendothelial cell migration, and in particular leukocyte extravasation, is an essential requirement in terms of controlling the passage of leukocytes from the circulation to the tissues both in terms of normal physiology and in the context of many unwanted pathologies. For example, under normal physiological conditions, monocytes and other leukocytes leave the circulation in order to circulate to the tissues. With respect to monocytes, in particular, tissue bound monocytes differentiate to macrophages. In another example, chronic inflammatory states such as rheumatoid arthritis and atherosclerosis are characterised by vessel hyper-permeability which allows increased transmigration of inflammatory cells across the activated endothelium. Accordingly, the present invention is particularly useful, but in no way limited to, use as a therapy to downregulate cellular transendothelial cell migration permeability where an individual is suffering from an unwanted inflammatory condition. Alternatively, the upregulation of cellular transendothelial cell migration may be desirable where it is necessary that passage of leukocytes, in particular neutrophils, is facilitated from the circulation into the tissue, such as for the purpose of facilitating a non-specific immune response to a pathogen localised in the tissue (e.g. treatment of infection).

The present invention therefore contemplates a method for the treatment and/or prophylaxis of a condition characterised by aberrant, unwanted or otherwise inappropriate cellular transendothelial cell migration in a mammal, said method comprising modulating the functional activity of ERK wherein upregulating ERK activity to a functionally effective level upregulates said cellular transendothelial cell migration and down-regulating ERK activity to a functionally ineffective level downregulates said cellular transendothelial cell migration.

Preferably, said endothelial cells are vascular endothelial cells and said cellular transendothelial cell migration is leukocyte extravasation. Most preferably, said leukocyte extravasation is neutrophil extravasation.

Reference to “aberrant, unwanted or otherwise inappropriate” cellular transendothelial cell migration should be understood as a reference to under-active migration, to physiologically normal migration which is inappropriate in that it is unwanted or to over-active migration As detailed hereinbefore, there are a number of conditions which are dependent on the induction of the correct level of cellular transendothelial cell migration, and in particular neutrophil extravasation. For instance, and in relation to the preferred embodiments disclosed herein, in individuals experiencing an unwanted inflammatory response, the downregulation of ERK to a functionally ineffective level provides a means for this unwanted inflammatory response to be retarded. This is of particular significance in the context of conditions such as atheromas, rheumatoid arthritis and inflammatory bowel disease. Upregulation of ERK activity may be desired in the context of treating unwanted pathogens or infection.

In a most preferred embodiment, there is provided a method for the treatment and/or prophylaxis of a condition characterised by unwanted cellular transendothelial cell migration in a mammal, said method comprising administering to said mammal an effective amount of an agent for a time and under conditions sufficient to induce a functionally ineffective level of ERK.

Preferably, said endothelial cells are vascular endothelial cells and said cellular transendothelial cell migration is leukocyte extravasation. More preferably, said leukocyte extravasation is neutrophil extravasation. Most preferably, said condition is an inflammatory condition.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject ERK may be administered together with an agonistic agent in order to enhance its effects. Alternatively, in the case of autoimmune inflammation, the ERK antagonist may be administered together with immunosuppressive drugs. By “sequential” administration is meant a time difference of from seconds, minutes, hours or days between the administration of the two types of molecules. These molecules may be administered in any order.

Another aspect of the present invention relates to the use of an agent capable of modulating the functionally effective level of ERK in the manufacture of a medicament for the regulation of cellular transendothelial cell migration in a mammal wherein upregulating ERK activity to a functionally effective level upregulates said cellular transendothelial cell migration and downregulating ERK activity to a functionally ineffective level downregulates said cellular transendothelial cell migration.

In another aspect the present invention relates to the use of ERK or a nucleic acid encoding ERK in the manufacture of a medicament for the regulation of cellular transendothelial cell migration wherein upregulating ERK to a functionally effective level upregulates said cellular transendothelial cell migration.

According to these preferred embodiments, said endothelial cells are preferably vascular endothelial cells and said cellular transendothelial cell migration is preferably leukocyte extravasation. More preferably, said leukocyte extravasation is neutrophil extravasation. Most preferably, said functioning is down-regulated.

The term “mammal” and “subject” as used herein includes humans, primates, livestock animals (e.g. sheep, pigs, cattle, horses, donkeys), laboratory test animals (e.g. mice, rabbits, rats, guinea pigs), companion animals (e.g. dogs, cats) and captive wild animals (e.g. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding ERK or a modulatory agent as hereinbefore defined. The vector may, for example, be a viral vector.

The present invention is further defined by the following non-limiting Examples.

### EXAMPLE 1

### Activation of Endothelial ERK is Essential for Neutrophil Transmigration: Potential Involvement of a Soluble Neutrophil Factor in Endothelial Activation

### Materials and Methods

Reagents and Antibodies

Chemically synthesized interleukin 8 (IL-8) was produced as a 72 amino acid form using automated solid phase methods. N-formyl-methionyl-leucinyl-phenylalanine (fMLP) was from Sigma (St. Louis, Wash.), recombinant human tumor necrosis factor-α (TNFα) was from Genentech (South San Francisco, Calif.; batch number 3056-55) or purchased from R & D Systems, Inc. (Minneapolis, Minn.). Purified human fibronectin (Boehringer Mannheim) diluted in phosphate buffered saline (PBS), pH 7.3, to 50 μg/ml was used for coating surfaces unless otherwise stated. Anhydrous cell culture grade DMSO (Sigma, St. Louis, Wash.) was used as solvent for PD98059. The inhibitors that act on MEK PD98059 were from Calbiochem (San Diego, Calif.) and U0126 was from Promega (Madison, Wis.). Phospho-ERK Ab was obtained from Promega (Madison, Wis.).

Culture of HUVEC

HUVEC were extracted by collagenase treatment according to a modified version of Wall et al., 1978, J. Cell Physiol. 96:203-213. Cells were grown in 25 cm² gelatin-coated tissue culture flasks (Costar, Cambridge, Mass.) in endotoxin-free M 199 medium (Cytosystems, Sydney, Australia) supplemented with 20% FCS (PA Biological, Sydney, Australia), 20 mm HEPES, sodium pyruvate and non-essential amino acids at 37° C. in a 5% CO₂ atmosphere. Cells were re-plated 2-5 days after establishment of culture by harvesting with 0.05% trypsin-0.02% EDTA. Endothelial cell growth supplement (Multicel, Trace Biosystems, Australia) at 25 mg/ml and heparin were added to cells that were passaged twice or more. In general, cells between passages 2 and 5 were used. All reagents used in the growth and passaging of HUVEC were made up under endotoxin-free conditions and contained between 10-100 pg/ml endotoxin determined by the Limulus amoebocyte assay.

Purification of Human Neutrophils

Neutrophils were purified from normal donors as previously described (Smith, W. B., J. R. Gamble, I. Clark-Lewis, and M. A. Vadas. 1991. Immunology 72:65-72) by dextran sedimentation followed by density gradient centrifugation with Lymphoprep (Nycomed, Oslo, Norway) and hypotonic lysis of erythrocytes. They were resuspended in assay medium (RPMI-1640 with 10 mM HEPES and 2.5% FCS) prior to use. Cytological examination of stained cytocentrifuged preparations showed >95% of the cells were neutrophils. Trypan blue staining confirmed over 98% of these cells were viable.

Transmigration Assay

This was performed as previously described using Transwells (6.5 mm diameter, 3 μM pore size, Costar, Cambridge, Mass.) on 24 well culture trays (Smith et al., 1991, supra). Briefly, 5×10⁴ HUVEC (between passages 2 and 5) were seeded in the upper chamber of each Transwell pre-coated with fibronectin (50 μg/mL for 30 minutes) and the cells were grown at 37° C. in 5% CO₂ supplemented air to form a confluent monolayer. Neutrophils were added at 5×10⁵ cells/well to the top chamber and where indicated chemoattractant was added to the lower chamber. The neutrophils were incubated at 37° C. for 1 h after which the number that had transmigrated into the lower chamber were collected and counted. Transmigration is expressed as a percentage of neutrophils added.

Neutrophils were counted using one of two methods. Neutrophils retrieved from the lower compartment were either counted directly using a Coulter counter (Model ZF, Coulter, Herts, UK) or using an indirect colourimetric assay based on the conversion of a tetrazolium salt (MTT) to a formazan. Briefly, MTT (0.2 mg/ml, Sigma, St. Louis, Wash.) was added to the lower chamber and incubated for 4 hours at 37° C. The neutrophils were pelleted by centrifugation, the pellets resuspended in 200 μl acid isopropanol for an hour and the absorbance at 550 nm determined. A standard curve was constructed by serial dilution of the neutrophil preparation and the percentage of neutrophils transmigrating was calculated from this. The two methods used produced results of good fit (least squares fit regression analysis, >95% confidence) (data not shown).

Chemotaxis Assay

Chemotaxis assays were performed using Transwells essentially as in the transmigration assay except that the HUVEC monolayer was omitted. In addition, instead of pre-coating the upper chamber with fibronectin, the lower chamber was pre-coated with gelatin to prevent adhesion of neutrophils. Assay medium with or without added chemoattractants were added to the lower chamber and 5×10⁵ neutrophils/well were placed into the upper chamber of the Transwells. Neutrophils that had migrated through the filters after 1 hour incubation at 37° C. were counted. Counts are expressed as a percentage of the total number of cells added.

Adhesion Assays

Adhesion assays were performed as previously described (Gamble, J. R., Y. Khew-Goodall, and M. A. Vadas. 1993. J Immunol. 150:4494-4503) with the exception that neutrophils were used. Briefly, HUVEC were seeded on fibronectin (50 μg/ml)-coated 96-well flat bottom plates at 5×10⁴ cells/well and cultured for 2 days as described above. After washing, neutrophils (5×10⁵/well) were added to the confluent HUVEC monolayer and incubated for 30 min at 37° C. in 5% CO₂ supplemented air, after which non-adherent neutrophils were gently washed off. After washing the cells were stained with Rose Bengal and total numbers of adherent neutrophils determined by densitometry. The number of adherent neutrophils was computed from a standard curve and expressed as a percentage of the neutrophils added.

In some cases where the endothelial monolayer was omitted, the neutrophils were plated directly onto 96-well tissue culture dishes.

ERK Activation Assay

HUVEC (10⁶ cells/well) were seeded in 6-well tissue culture dishes. The confluent monolayers, either untreated or pre-treated as indicated, were washed in phosphate buffered saline and lysed in 20 mM Tris-Cl, pH 8.0 containing 150 mM NaCl, 1 mM CaCl, 1% Triton-X 100, 5 mM leupeptin, 10 mM PMSF, 25 mM benzamidine, 50 mM Na fluoride, 1 mM Na vanadate, and 50 mM β-glycerophosphate (all from Sigma, St. Louis, Wash.) for western blotting analysis. Protein concentration was determined using the Bradford reagent (BioRad) and equal amounts of protein loaded onto a 7.5% SDS polyacrylamide gel. Western blots were carried out using an antibody specific to phosphorylated ERK, ie. the activated form of ERK, and developed by enhanced chemiluminescence (Amersham). Total ERK present was determined by stripping the filter and re-blotting with an antibody against ERK 1/2.

### EXAMPLE 2

### Neutrophil Transmigration across Endothelium is Inhibited by Inhibitors that act on MEK

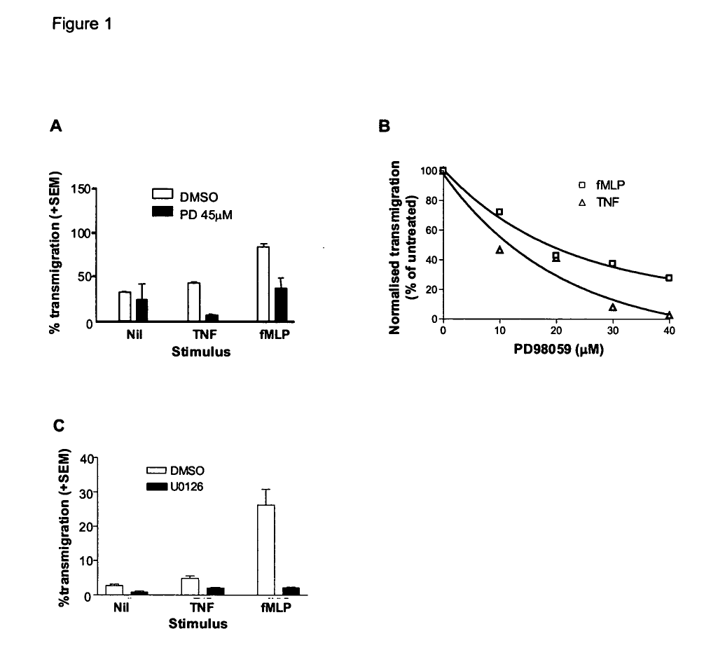

To identify signalling pathways in the endothelium that are essential for neutrophil transmigration, a screen for their effects on transmigration was carried out using pharmacological inhibitors of various signalling pathways (data not shown). The studies were performed using an in vitro model of neutrophil transmigration across a confluent monolayer of cultured human umbilical vein endothelial cells (HUVEC). Transmigration induced by an exogeneously added chemoattractant gradient and across TNFα-activated endothelium were both examined. PD98059, an inhibitor that acts on MEK, the upstream activator of the extracellular regulated kinases (ERK) 1/2, was found to inhibit in a dose-dependent manner neutrophil transmigration induced by a chemoattractant (fMLP) gradient as well as transmigration across TNFα-activated endothelium (FIGS. 1A, B). In general, transmigration across TNFα-activated endothelium was inhibited to a greater extent than transmigration across a gradient of fMLP, with 70-80% inhibition of transmigration across TNFα-activated endothelium compared to only 40-50% inhibition of transmigration across an fMLP gradient. The inhibitory effects of PD98059 were also confirmed using a second MEK inhibitor, U0126 (FIG. 1C). Interestingly, although U0126 inhibited transmigration across TNFα-activated endothelium to a similar extent as PD 98059, it showed greater potency against fMLP-driven transmigration.

### EXAMPLE 3

### PD 98059 DOES NOT INHIBIT Transmigration by Inhibiting Neutropril Chemotaxis or Adhesion

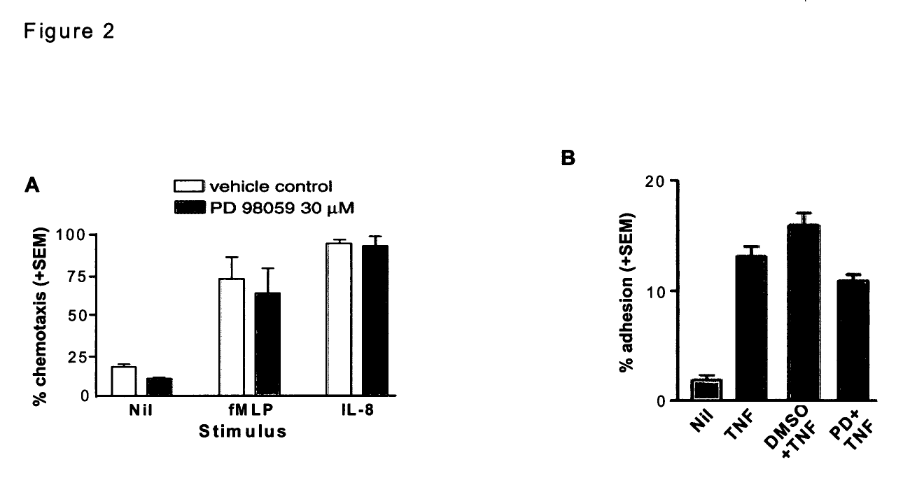

Two important properties of leukocytes governing their ability to transmigrate across an endothelial barrier are their ability to migrate and to adhere to the endothelium. Because the neutrophils were exposed to the MEK inhibitor throughout the duration of the transmigration assay, the effect of PD98059 on neutrophil migration and adhesion were both examined. To investigate the effect of PD98059 on neutrophil migration, a chemotaxis assay (in the absence of an endothelial monolayer) across a chemoattractant gradient was used. In addition to fMLP, IL-8 was also used as a chemoattractant to mimic the resultant IL-8 chemoattractant gradient generated when TNFα-activated endothelium is used in the transmigration assay (Smith, W. B., J. R. Gamble, I. Clark-Lewis, and M. A. Vadas. 1993. Immunology 78:491-497). Both fMLP- and IL-8-stimulated neutrophil chemotaxis were not significantly affected by PD 98059 when it was included with the assay (FIG. 2A). This suggests that the inhibitor had no effect on the ability of neutrophils to sense a chemotactic gradient or their ability to migrate towards it.

To determine whether exposure of neutrophils to PD98059 affected neutrophil adhesion to TNFα-activated endothelium, an adhesion assay was carried out on TNFα-activated endothelium in the presence of the inhibitor or its vehicle. TNFα treatment of endothelium stimulated neutrophil adhesion but adhesion was not significantly affected by the inclusion of PD98059 (FIG. 2B), suggesting that the inhibitor did not affect the ability of neutrophils to adhere to the endothelium.

### EXAMPLE 4

### Endothelial ERK is Activated by Neutrophils

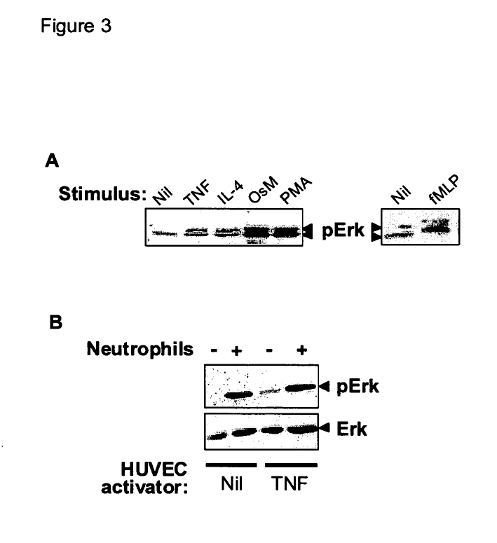

Data presented in FIG. 2 suggested that the decreased transmigration caused by inhibitors of ERK activation was not due to their effect on neutrophil function per se. This, in turn, suggested that endothelial ERK activation may be essential for transmigration to occur. We therefore investigated whether ERK activation in the endothelium may be occurring under the conditions of the transmigration assay and which parameter(s) present in the assay system were responsible for its activation. Initially, the role of the inducers, TNFα and fMLP, used in the transmigration assay were assessed. Endothelial monolayers were treated with TNFα or fMLP, as well as a number of cytokine and non-cytokine activators of the endothelium, and ERK activation determined by Western blotting with an Ab specific for the MEK-phosphorylated form of ERK, ie. activated ERK. fMLP did not activate endothelial ERK (FIG. 3A, right panel). TNFα and IL-4 marginally activated ERK (FIG. 3A, left panel). This is consistent with our earlier observations showing a 1.5-2.0-fold activation of ERK (Xia, P., J. R. Gamble, K. A. Rye, L. Wang, C. S. Hii, P. Cockerill, Y. Khew-Goodall, A. G. Bert, P. J. Barter, and M. A. Vadas. 1998. Proc Natl Acad Sci USA 95:14196-14201). However, the degree of ERK activation was small in comparison to other activators such as OsM and PMA (FIG. 3A). Furthermore, because both fMLP-(which did not activate ERK) and TNFα-induced transmigration were inhibited by PD98059, we explored the possibility that the endothelial ERK activation occurring during transmigration might be triggered by other factors.

The role of the neutrophils in activating endothelial ERK was therefore investigated. An endothelial monolayer was set up as in the transmigration assay. The endothelial monolayer was either pre-stimulated with TNFα or left unstimulated with fMLP included at the time of neutrophil addition. Following incubation with the endothelial monolayer, neutrophils were removed and the endothelial monolayer washed extensively to ensure complete removal of neutrophils prior to lysis and Western blotting with the phospho-ERK Ab to detect activated ERK. Addition of neutrophils to both resting and TNFα-activated endothelium resulted in a dramatic increase in ERK activation (FIG. 3B), much greater (at least 10-fold) than that observed when TNFα was added alone. In addition, there was, no significant difference in the degree of ERK activation between unstimulated and TNFα-stimulated endothelium, indicating that pre-activation of the endothelium by TNFα was not necessary for the neutrophils to trigger endothelial ERK activation. The increase in phospho-ERK following incubation with neutrophils is unlikely to be due to contaminating neutrophils because analysis of the neutrophils that were removed showed no ERK activation (data not shown).

### EXAMPLE 5

### Neutrophil Adhesion to Endothelium is not Essential for Activation of Endothelial ERK

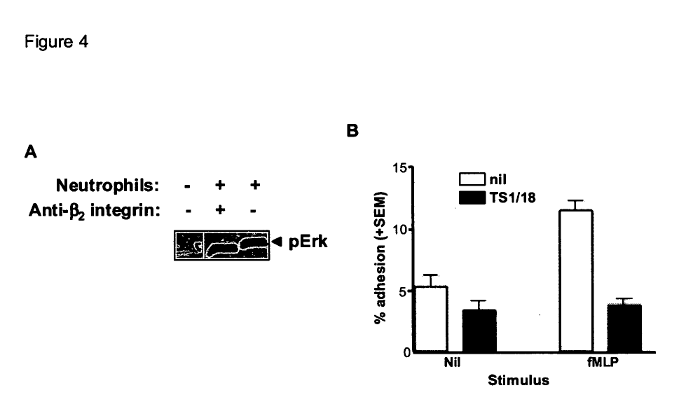

A crucial step in leukocyte extravasation is firm adhesion of the leukocytes to the endothelium mediated by the binding of the leukocyte β₂ (CD18) integrins to their receptors on the endothelium (reviewed in Butcher et al. 1991, supra; Springer et al., 1994, supra). To investigate whether adhesion to the endothelium was essential for activation of endothelial ERK by neutrophils, the neutrophils were either pre-incubated with a functional blocking Ab to β₂ integrin (TS 1/18) or not before being added to the endothelial monolayer in the presence of fMLP. After 15 min, the neutrophils and media were removed, the endothelial monolayer washed and lysed. Western blots to detect ERK activation showed that ERK activation occurred only in the presence of neutrophils as expected and was not reduced by pre-treatment with the functional blocking anti-β₂ integrin Ab (FIG. 4A). Parallel adhesion assays carried out in the presence or absence of fMLP confirmed that pre-treatment of neutrophils with the anti-β₂ integrin Ab did indeed block fMLP-stimulated neutrophil adhesion but not to basal adhesion (FIG. 4B). These data indicate that neutrophil-stimulated ERK activation was not dependent on β₂ integrin-mediated adhesion.

### EXAMPLE 6

### A Soluble Neutrophil Factor Activates Endothelial ERK

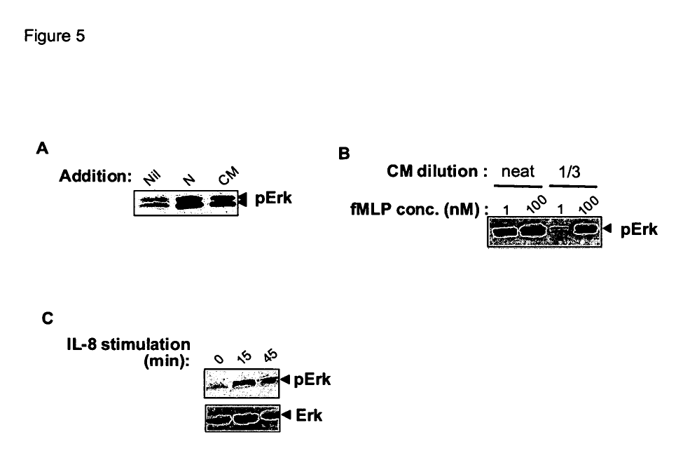

Because neutrophil adhesion to the endothelium was not essential for endothelial ERK activation, the possibility that the inducer may be a soluble factor produced by the neutrophils was examined. Conditioned medium from neutrophils stimulated with fMLP was harvested and used to stimulate endothelial monolayers. Care was taken to ensure that no neutrophils were carried over into the supernatants. As controls, an equivalent proportion of the neutrophils used to generate the conditioned medium as well as medium with only fMLP added were used to stimulate endothelial monolayers. fMLP alone had no effect on endothelial ERK activation but both the neutrophils and conditioned medium activated ERK (FIG. 5A), suggesting that a soluble neutrophil factor is the active agent in activating endothelial ERK. ERK activation induced by the conditioned medium was less than that induced when neutrophils were added but was still significantly greater than the basal level of activated ERK present in resting endothelium or endothelium treated with fMLP alone. This may be attributed to the possibility that the neutrophils were continuing to produce the factor during the period of incubation with HUVEC, resulting in a local higher concentration of factor than when only the conditioned medium was added. The dosage of fMLP required to induce production of the factor and the dosage of conditioned medium required to activate endothelial ERK were also investigated. Neutrophil conditioned media were prepared after incubation of neutrophils with 1 or 100 nM fMLP. At each concentration of conditioned medium (neat or one-third dilution) added to HUVEC, increasing the fMLP concentration used to stimulate neutrophils from 1 nM to 100 nM led to an increase in the ERK-activating factor produced; the effect of fMLP dosage is more marked at the lower concentration of conditioned medium used (FIG. 5B). At each fMLP concentration used to induce the ERK-activating factor, increasing the amount of conditioned medium added to HUVEC resulted in an increase in endothelial ERK activation, with the dose-dependency being more marked when the lower fMLP concentration was used to induce the ERK-activating factor (FIG. 5B).

TNFα activation of endothelial cells results in the production of IL-8. We therefore investigated whether neutrophils exposed to IL-8 would also be induced to produce the ERK-activating factor. Incubation of neutrophils with IL-8, like fMLP, also induced the ERK-activating factor (FIG. 5C).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Allport, J. R., Ding, H., Collins, T., Gerritsen, M. E. and Luscinskas, F. W. (1997). Endothelial-dependent mechanisms regulate leukocyte transmigration: a process involving the proteasome and disruption of the vascular endothelial-cadherin complex at endothelial cell-to-cell junctions. J Exp. Med. 186:517-527
Allport, J. R., Muller, W. A. and Luscinskas, F. W. (2000). Monocytes induce reversible focal changes in vascular endothelial cadherin complex during transendothelial migration under flow. J Cell Biol 148:203-216
Altschul, et al. (1990) J. Mol. Biol. 215:403-410
Altschul et al. (1997) Nucleic Acids Res. 25:3389-3402
Bianchi, E., Bender, J. R., Blasi, F. and Pardi, R. (1997). Through and beyond the wall: late steps in leukocyte transendothelial migration. Immunol. Today 18:586-591
Bunin B A, et al. (1994) Proc. Natl. Acad. Sci. USA, 91:4708-4712.
Butcher, E. C. (1991). Leukocyte-endothelial cell recognition: three (or more) steps to specificity and diversity. Cell 67:1033-1036.
Cepinskas, G., Noseworthy, R. and Kvietys, P. R. (1997). Transendothelial neutrophil migration. Role of neutrophil-derived proteases and relationship to transendothelial protein movement. Circ. Res. 81:618-626
Cepinskas, G., Sandig, M. and Kvietys, P. R. (1999). PAF-induced elastase-dependent neutrophil transendothelial migration is associated with the mobilization of elastase to the neutrophil surface and localization to the migrating front. J Cell Sci 112 (Pt 12): 1937-1945
Dejana, E., Corada, M. and Lampugnani, M. G. (1995). Endothelial cell-to-cell junctions. FASEB J9:910-918
Dejana, E., Lampugnani, M. G., Martinez-Estrada, 0 and Bazzoni, G. (2000). The molecular organization of endothelial junctions and their functional role in vascular morphogenesis and permeability. Int. J. Dev. Biol. 44:743-748
Del Maschio, A., Zanetti, A., Corada, M., Rival, H., Ruco, L., Lampugnani, M. G. and Dejana E. (1996). Polymorphonuclear leukocyte adhesion triggers the disorganization of endothelial cell-to-cell adherens junctions. J Cell Biol 135:497-510
DeWitt S H, et al. (1993) Proc. Natl. Acad. Sci. USA, 90:6909-6913.
Dvorak, H. F., Brown, L. F., Detmar, M., Dvorak, A. M. (1995) Am J Pathol. 146:1029-39.
Dvorak, H. F., Harvey, V. S., Estrella, P., Brown, L. F., McDonagh, J., Dvorak, A. M. (1987) Lab Invest. 57:673-86.
Gamble, J. R., Y. Khew-Goodall, and M. A. Vadas. (1993). Transforming growth factor-beta inhibits E-selectin expression on human endothelial cells. J Immunol. 150:4494-4503.
Hixenbaugh, E. A., Goeckeler, Z. M., Papaiya, N. N., Wysolmerski, R. B., Silverstein, S. C. and Huang, A. J. (1997). Stimulated neutrophils induce myosin light chain phosphorylation and isometric tension in endothelial cells. Am. J Physiol 273:H981-H988
Hoshi, M., Ohta, K., Gotoh, Y., Mori, A., Murofushi, H., Sakai, H. and Nishida, E. (1992). Mitogen-activated-protein-kinase-catalyzed phosphorylation of microtubule-associated proteins, microtubule-associated protein 2 and microtubule-associated protein 4, induces an alteration in their function. Eur. J. Biochem 203:43-52
Huang, A. J., Manning, J. E., Bandak, T. M., Ratau, M. C., Hanser, K. R. and Silverstein, S. C. (1993). Endothelial cell cytosolic free calcium regulates neutrophil migration across monolayers of endothelial cells. J Cell Biol 120:1371-1380
Karlin and Altschul (1990) Proc. Natl. Acad. Sci. USA 87:2264-2268
Karlin and Altschul (1993) Proc. Natl. Acad. Sci. USA 90:5873-5877
Lampugnani, M. G. and Dejana, E. (1997) Interendothelial junctions: structure, signalling and functional roles. Curr. Opin. Cell Biol 9:674-682
Muller, W. A. and Randolph, G. J. (1999). Migration of leukocytes across endothelium and beyond: molecules involved in the transmigration and fate of monocytes. J Leukoc. Biol 66:698-704
Muller, W. A., Weigl, S. A., Deng, X. and Phillips D. M. (1993). PECAM-1 is required for transendothelial migration of leukocytes. J Exp. Med. 178:449-460
Myers and Miller, CABIOS (1989)
Nakada M. T., Amin, K., Christofidou-Solomidou, M., O'Brien, C. D., Sun, J., Gurubhagavatula, I., Heavner, G. A., Taylor, A. H., Paddock, C., Sun, Q. H., Zehnder, J. L., Newman, P. J., Albelda, S. M. and DeLisser, H. M. (2000). Antibodies against the first Ig-like domain of human platelet endothelial cell adhesion molecule-1 (PECAM-1) that inhibit PECAM-1-dependent homophilic adhesion block in vivo neutrophil recruitment. J Immunol. 164:452-462
Newman, P. J. (1997). The biology of PECAM-1. J. Clin. Invest 100:S25-S29
Saito, H., Minamiya, Y., Kitamura, M., Saito, S., Terada, K. and Ogawa, J. (1998). Endothelial myosin light chain kinase regulates neutrophil migration across human umbilical vein endothelial cell monolayer. J Immunol. 161:1533-1540
Schlesinger, T. K., Fanger, G. R., Yujiri, T. and Johnson, G. L. (1998). The TAO of MEKK. Front Biosci 3:D1181-D1186
Shaw, S. K., Bamba, P. S., Perkins, B. N. and Luscinskas, F. W. (2001). Real-time imaging of vascular endothelial-cadherin during leukocyte transmigration across endothelium. J Immunol. 167:2323-2330
Smith, W. B., J. R. Gamble, I. Clark-Lewis, and M. A. Vadas. (1991). Interleukin-8 induces neutrophil transendothelial migration. Immunology 72:65-72.
Smith, W. B., J. R. Gamble, I. Clark-Lewis., and M. A. Vadas. (1993). Chemotactic desensitization of neutrophils demonstrates interleukin-8 (IL-8)-dependent and IL-8-independent mechanisms of transmigration through cytokine-activated endothelium. Immunology 78:491-497.
Springer, T. A. (1994). Traffic signals for lymphocyte recirculation and leukocyte emigration: the multistep paradigm. Cell 76:301-314.
Su, W. H., Chen, H. I. and Jen, C. J. (2002). Differential movements of VE-cadherin and PECAM-1 during transmigration of polymorphonuclear leukocytes through human umbilical vein endothelium. Blood 100:3597-3603
Su, W. H., Chen, H. I., Huang, J. P. and Jen, C. J. (2000). Endothelial [Ca(2+)](i) signaling during transmigration of polymorphonuclear leukocytes. Blood 96:3816-3822
Wall, R. T., L. A. Harker, L. J. Quadracci, and G. E. Striker. (1978). Factors influencing endothelial cell proliferation in vitro. J. Cell Physiol. 96:203-213.
Xia, P., J. R. Gamble, K. A. Rye, L. Wang, C. S. Hii, P. Cockerill, Y. Khew-Goodall, A. G. Bert, P. J. Barter, and M. A. Vadas. (1998). Tumor necrosis factor-alpha induces adhesion molecule expression through the sphingosine kinase pathway. Proc Natl Acad Sci USA 95:14196-14201.

1. A method of modulating mammalian cellular transendothelial cell migration, said method comprising modulating endothelial cell ERK functional activity wherein upregulating ERK activity to a functionally effective level upregulates said cellular transendothelial cell migration and downregulating said activity to a functionally ineffective level downregulates said cellular transendothelial cell migration.

2. A method of modulating cellular transendothelial cell migration in a mammal, said method comprising modulating endothelial cell ERK functional activity in said mammal wherein upregulating ERK activity to a functionally effective level upregulates said cellular transendothelial cell migration and downregulating ERK activity to a functionally ineffective level downregulates said cellular transendothelial cell migration.

3. A method for the treatment and/or prophylaxis of a condition characterised by aberrant, unwanted or otherwise inappropriate cellular transendothelial cell migration in a mammal, said method comprising modulating the functional activity of ERK wherein upregulating ERK activity to a functionally effective level upregulates said cellular transendothelial cell migration and downregulating ERK activity to a functionally ineffective level downregulates said cellular transendothelial cell migration.

4. The method according to any one of claims 1-3, wherein said endothelial cell is a vascular endothelial cell.

5. The method according to any one of claims 1-3 wherein said transendothelial cell migration is extravasation.

6. The method according to claim 5 wherein said cellular extravasation is leukocyte extravasation.

7. The method according to claim 6 wherein said leukocyte is a neutrophil.

8. The method according to claim 5 wherein said transendothelial cell migration is modulated by downregulation of extravasation.

9. The method according to claim 5 wherein said transendothelial cell migration is modulated by upregulation of extravasation.

10. The method according to claim 3 wherein said condition is an unwanted inflammatory condition, said endothelial cell is a vascular endothelial cell and said cellular transendothelial cell migration is leukocyte extravasation which is down-regulated.

11. The method according to claim 10 wherein said leukocyte is a neutrophil.

12. The method according to claim 111 wherein said inflammatory condition is rheumatoid arthritis, atherosclerosis or inflammatory bowel disease.

13. The method according to claim 12 wherein said rheumatoid arthritis, atherosclerosis or inflammatory bowel disease are chronic.

14. The method according to claim 3 wherein said condition is an infection, said endothelial cell is a vascular endothelial cell and said cellular transendothelial cell migration is leukocyte extravasation which is upregulated.

15. The method according to claim 14 wherein said leukocyte is a neutrophil.

16. The method according to claim 15 wherein said infection is a pathogen infection.

17. The method according to any one of claims 1-3 wherein said modulation is upregulation of ERK functional activity and said upregulation is achieved by introducing into said endothelial cell a nucleic acid molecule encoding ERK or functional equivalent, derivative or homologue thereof or the ERK expression product or functional derivative, homologue, analogue, equivalent or mimetic thereof.

18. The method according to any one of claims 1-3 wherein said modulation is achieved by contacting said endothelial cell with a proteinaceous or non-proteinaceous molecule which modulates transcriptional and/or translational regulation of the ERK gene.

19. The method according to any one of claims 1-3 wherein said modulation is upregulation of ERK functional activity and said upregulation is achieved by contacting said endothelial cell with a proteinaceous or non-proteinaceous molecule which functions as an agonist of the ERK expression product.

20. The method according to any one of claims 1-3 wherein said modulation is downregulation of ERK functional activity and said downregulation is achieved by contacting said endothelial cell with a proteinaceous or non-proteinaceous molecule which functions as an antagonist to the ERK expression product.

21. The method according to claim 20 wherein said antagonist is a kinase inhibitor.

22. The method according to claim 21 wherein said kinase inhibitor is PD98059 or U0126 functional derivative or equivalent thereof.

23. The method according to claim 21 wherein said kinase inhibitor is PD184352 or functional derivative or equivalent thereof.

24. The method according to claim 20 wherein said antagonist is an anti-ERK antibody.

25. The method according to claim 19 wherein said agonist is an activator of ERK or kinases upstream from ERK.

26. The method according to claim 1 wherein said endothelial cell activity is modulated in vivo.

27. The method according to claim 1 wherein said endothelial cell activity is modulated in vitro.

28. Use of an agent capable of modulating the functionally effective level of ERK in the manufacture of a medicament for the regulation of cellular transendothelial cell migration in a mammal wherein upregulating ERK activity to a functionally effective level upregulates said cellular transendothelial cell migration and downregulating ERK activity to a functionally ineffective level downregulates said cellular transendothelial cell migration.

29. Use of an agent capable of modulating the functionally effective level of ERK in the manufacture of a medicament for the treatment of a condition characterised by aberrant, unwanted or otherwise inappropriate cellular transendothelial cell migration wherein upregulating ERK activity to a functionally effective level upregulates said cellular transendothelial cell migration and downregulating ERK activity to a functionally ineffective level downregulates said cellular transendothelial cell migration.

30. Use according to claim 28 or 29 wherein said endothelial cell is a vascular endothelial cell.

31. Use according to claim 28 or 29 wherein said transendothelial cell migration is extravasation.

32. Use according to claim 31 wherein said cellular extravasation is leukocyte extravasation.

33. Use according to claim 32 wherein said leukocyte is a neutrophil.

34. Use according to claim 31 wherein said transendothelial cell migration is modulated by downregulation of extravasation.

35. Use according to claim 31 wherein said transendothelial cell migration is modulated by upregulation of extravasation.

36. Use according to claim 29 wherein said condition is an unwanted inflammatory condition, said endothelial cell is a vascular endothelial cell and said cellular transendothelial cell migration is leukocyte extravasation which is down-regulated.

37. Use according to claim 36 wherein said leukocyte is a neutrophil.

38. Use according to claim 37 wherein said inflammatory condition is rheumatoid arthritis, atherosclerosis or inflammatory bowel disease.

39. Use according to claim 38 wherein said rheumatoid arthritis, atherosclerosis or inflammatory bowel disease are chronic.

40. Use according to claim 29 wherein said condition is an infection, said endothelial cell is a vascular endothelial cell and said cellular transendothelial cell migration is leukocyte extravasation which is upregulated.

41. Use according to claim 40 wherein said leukocyte is a neutrophil.

42. Use according to claim 41 wherein said infection is a pathogen infection.

43. Use according to claim 28 or 29 wherein said modulation is upregulation of ERK functional activity and said upregulation is achieved by introducing into said endothelial cell a nucleic acid molecule encoding ERK or functional equivalent, derivative or homologue thereof or the ERK expression product or functional derivative, homologue, analogue, equivalent or mimetic thereof.

44. Use according to claim 28 or 29 wherein said modulation is achieved by contacting said endothelial cell with a proteinaceous or non-proteinaceous molecule which modulates transcriptional and/or translational regulation of the ERK gene.

45. Use according to claim 28 or 29 wherein said modulation is upregulation of ERK functional activity and said upregulation is achieved by contacting said endothelial cell with a proteinaceous or non-proteinaceous molecule which functions as an agonist of the ERK expression product.

46. Use according to claim 28 or 29 wherein said modulation is downregulation of ERK functional activity and said downregulation is achieved by contacting said endothelial cell with a proteinaceous or non-proteinaceous molecule which functions as an antagonist to the ERK expression product.

47. Use according to claim 46 wherein said antagonist is a kinase inhibitor.

48. Use according to claim 47 wherein said kinase inhibitor is PD98059 or U0126 or functional derivative or equivalent thereof.

49. Use according to claim 47 wherein said kinase inhibitor is PD184352 or functional derivative or equivalent thereof.

50. Use according to claim 46 wherein said antagonist is an anti-ERK antibody.

51. Use according to claim 45 wherein said agonist is an activator of ERK or kinases upstream from ERK.

52. A pharmaceutical composition comprising the modulatory agent as hereinbefore defined and one or more pharmaceutically acceptable carriers and/or diluents.
